(19) United States
(12) Patent Application Publication
(10) Pub. No.: US 2024/0316279 A1
(43) Pub. Date: Sep. 26, 2024

(54) Pre-filled syringe (PFS) with twist-breakable tip, ensuring that the syringe is tamper-resistant and can be reliably opened

(71) Applicant: LABORATOIRE AGUETTANT, LYON (FR)

(72) Inventors: Jeff TONNAR, LYON (FR); Vincent GUYOT WITTMANN, MUNICH (DE); Cédric PENARD, TASSIN LA DEMI-LUNE (FR)

(21) Appl. No.: 18/038,982

(22) PCT Filed: Nov. 26, 2021
(86) PCT No.: PCT/EP2021/083144

§ 371 Date: May 25, 2023

(30) Foreign Application Priority Data
Nov. 26, 2020 (FR) 2012159

(51) Int. Cl. A61M 5/28

(52) U.S. Cl. CPC A61M 5/285

(57) ABSTRACT

A pre-filled syringe (1) has a cap (14) which is snap-fastened/click-fitted onto the connecting tip (9) or the tubular body of the syringe, and inside which a breakable plug (13) is secured or connected. The plug is guaranteed to rupture when at least one portion of the cap is twisted about the longitudinal axis (X) of the syringe.

### PRIOR ART

The present invention relates to the field of the packaging of injectable medicinal products for parenteral administration in liquid form.

More specifically, the invention relates to pre-filled syringes (PFS) in which medicinal products are packaged and which require aseptic presentation, which is to say that the interface for the transfer of the medicinal products to the point at which they are being administered (the injection site in a patient) has to be sterile at the moment of use.

The invention seeks to offer a twist-breakable tip which ensures both tamper-resistance and reliable opening of the syringe.

### PRIOR ART

A pre-filled syringe is a syringe in which a medicinal product in liquid form has been packaged and sterilized, before or after the filling of the syringe, so that it can be stored for what might be a long time. This makes it possible to avoid a step of withdrawing medicinal product from a bottle or an ampule at the moment of use, so that administration can be immediate.

This ready-use packaging avoids the need to prepare the medicinal product before injecting it, along with the associated risks of error.

In order to prevent the medicinal product from seeping out while it is being stored in the syringe, the latter is closed at one end of the volume containing the medicinal product by a seal mounted on the plunger of the syringe, and at the other end by a tip with a removable closure.

FIG. 1 depicts a pre-filled hypodermic syringe 1 according to the prior art, exhibiting symmetry of revolution about a longitudinal axis X.

The syringe 1 comprises a tubular body 2 of cylindrical overall shape with two open longitudinal ends 3, 4.

A plunger 5 passes through the opening of the first end 3 of the tubular body 2. The end of the plunger 5 that is mounted inside the tubular body 2 is surmounted by a seal 6. The plunger 5 and the seal 6 form a piston able to slide inside the tubular body 2 along the longitudinal axis X. The seal 6 is preferably provided with peripheral ribs in direct surface contact, with elastic deformation, against the interior of the body 2 in order to ensure fluid tightness.

The plunger 5 allows a user to bring about the translation movement of the piston in order to expel the packaged liquid, once the syringe has been opened.

The tubular body 2 comprises, at the first end 3, a finger grip 7 which projects toward the outside. Thus, some of the digits of a user press against the underside of the finger grip 7, while another digit, generally the thumb, moves the plunger 5 and therefore the piston with its seal 6 by pressing on the end 50 of the plunger 5 which is the opposite end to the end that forms the piston.

The second end 4 of the tubular body comprises a substantially transverse wall 8 that is open at its center and extended by a connection tip 9 of the Luer lock or Luer or NR Fit or EN Fit type (or any other form of connection specifically developed for specific applications enabling errors with the other existing forms of connection to be avoided).

The tip 9 comprises a tubular interior part 10 and an exterior part 11, coaxial with the interior part, which forms a sleeve and the internal surface of which is threaded so that a tip of complementing shape can be screwed in to connect it to a needle or a perfusion line or a catheter or a device known as a Luer Activated Device (LAD) or any other commercially available connector once the syringe 1 has been opened.

The tubular interior part 10 is of substantially frustoconical shape and opens, at its larger-diameter end that coincides with the opening in the transverse wall 8, onto the inside of the tubular body 2.

A closure 12 is screwed into the threaded part 11 of the tip 9, thereby stoppering the tubular interior part 10 of the tip 9.

The tubular body 2, the piston with its seal 6, the tip 9 and the closure 12 delimit a fluid tight space which acts as a reservoir for a liquid that is to be administered to a patient.

In order to render the syringe 1 operational, the closure 12 is unscrewed, and this opens the syringe, and then a needle or a perfusion line or a catheter or an LAD or some other commercially available connector, is connected to the tip 9.

Once the needle or the perfusion line or the catheter or other commercially available connector has been implanted in a patient, a user can actuate the plunger 5 and this will administer to the patient the liquid contained in the reservoir.

The overriding advantage of a pre-filled syringe is that it avoids the user needing to transfer a medicinal product from some other container into the syringe prior to use. In other words, a pre-filled syringe limits the preparation, confusion and risk of contamination at the moment of injection, because all of the contact zones are sterile, insofar as the pre-filled syringe is inside a container (blister) that is sterile. Thus, a pre-filled syringe allows rapid and safe administration of the medicinal product. Such a syringe generally comes with a graduated scale allowing dosage as desired.

There are various closures and connection tips that have been proposed for pre-filled syringes.

Thus, for syringes having a tubular body and a connecting tip made of glass or of plastic, there are closures made of elastomer, like the one referenced 12 in FIG. 1, which are inserted or screwed around the cone of the tip.

For example, the closure may be made of polypropylene and screwed directly onto a tip of the Luer lock type which is either of one piece with or attached to the tubular body of the syringe.

One significant demand made of pre-filled syringes is that of making them tamper-resistant, which is to say of guaranteeing to a user that the syringe opened is actually still sterile at its final moment of use.

Closures/caps that incorporate a tamper-resistance function have therefore been developed in order to ensure the sterility of those parts of the syringe having a body made of plastic or of glass and which are protected by a closure/cap. Some of these closures/caps are configured in such a way that the user has visual proof of tampering (tamper evidence) when the syringe has been opened undesirably prior to its intended final use.

In order to overcome the imperfection of certain elastomer closures/caps which are not sufficiently fluid tight for sterilization and which are therefore liable to cause the contents of the syringe to be affected, the applicant company has, in patent application WO2007/083034, proposed a breakable stopper coupled to a cap the twisting of which allows it to be detached from the opening of the connection tip thus freeing access to the contents of the syringe. This breakable stopper therefore preserves the integrity of the inside of the syringe body without an elastomer closure. One potential disadvantage with this solution is that in certain circumstances, notably in emergencies, and even though this may arise only very infrequently, there are still risks of incorrect opening if the cap is not correctly twisted by the user, thus preventing correct opening and therefore use of the syringe.

There is therefore a need to offer a pre-filled syringe stoppering solution that offers both the advantages of a conventional closure/cap, namely essentially simple and effective opening of the syringe with it being mechanically impossible to open if the user does not perform the correct action, and of a breakable closure/cap with stopper, which is to say essentially a guarantee of the integrity of the contents of the syringe whatever the sterilization process and with also a tamper-resistance function.

It is an objective of the invention to at least partially address this need.

### SUMMARY OF THE INVENTION

In order to achieve this, one of the aspects of the invention relates to a pre-filled syringe comprising:
a tubular body extending along a longitudinal axis, having two open ends and containing a liquid to be administered to a patient,
a plunger one of the ends of which forms a piston mounted for sliding in the tubular body, the plunger passing through the opening of one of the ends of the tubular body,
a connection tip comprising a tubular part communicating with the opening of the other of the ends of the tubular body to create the passage for the fluid that is to be administered;
a fluid tight stopper comprising a breakable zone connected to the free end of the tubular part of the tip,
a cap comprising:
   a fixing or coupling portion for fixing or coupling to the stopper,
   a portion forming a skirt in dissociable form or molded as one with the fixing or coupling portion, and which extends as far as the tubular body;
      means for fixing at least part of the cap to the tubular body or the connecting tip, the means comprising at least one snap-fastening lug and at least one snap-fastening opening or groove collaborating in snap-fastening with the snap-fastening lug and being configured to allow a twisting of at least part of the cap about its longitudinal axis to cause the breakable zone to rupture thereby allowing the cap with the stopper to be physically separated from the tip.

Any other undesirable manual effort applied to part of the cap, other than a twisted action and, for example, a pulling action, will not allow the cap to be opened. Specifically, the lug or lugs individually snap-fastened into an opening mechanically prevent unwanted removal of the cap, which might be attempted notably by pulling on it.

According to a first advantageous configuration:
the connection tip comprises an exterior part, at the exterior periphery of the tubular part, comprising the at least one outwardly projecting lug;
the skirt comprises the at least one open-ended opening or groove collaborating in snap-fastening with the at least one lug.

Alternatively, the lug(s) can be made on the inside of the cap with a complementing snap-fastening groove made in the connection tip or the tubular body of the syringe.

Thus, according to a second advantageous configuration:
the skirt comprises an interior part comprising the at least one inwardly projecting lug;
the connection tip comprises an exterior part, at the exterior periphery of the tubular part, the at least one open-ended opening or groove collaborating in snap-fastening with the at least one lug.

According to a first advantageous embodiment, the cap comprises:
two distinct parts one of them being in dissociable form or molded as one with the fixing or coupling portion and the other extending as far as the tubular body and incorporating the at least one opening or groove,
at least one twist-breakable bridging piece, connecting the two parts some distance from one another so that the twisting of the part in dissociable form or molded as one with the fixing or coupling portion does not cause the other part, which is kept snap-fastened, to turn but causes simultaneous rupturing of the breakable zone and of the at least one bridging piece, the rupturing of the latter providing the user with a visual indication that the action has been performed correctly.

In the first embodiment, the simultaneous breakage of the bridging piece or pieces and of the stopper provides the syringe with tamper resistance.

After the twisting action has caused the bridging piece or pieces to rupture, pulling the cap away from the body of the syringe allows the cap to be released with the stopper coupled to it and therefore allows access to the medicinal product contained in the syringe.

According to a second advantageous embodiment, the cap is produced as a one-piece single component comprising:
the at least one snap-fastening groove or opening delimited by at least two zones, a first in which the snap-fastening of the lug occurs and at least a second in the continuity of the first, and in which the lug becomes lodged when the cap is twisted;
at least one disengagement open-ended opening or groove in the continuation of the second zone, the disengagement open-ended opening or groove being configured to allow physical separation of the cap with the stopper.

According to this second embodiment and in a variant, the at least one snap-fastening groove or opening comprises a single second zone allowing the cap to be twisted in just one direction of rotation.

According to this second embodiment and another advantageous embodiment variant, the at least one snap-fastening groove or opening comprises a narrowing between the two zones, which narrowing is configured so that when the cap is twisted, the action of the lug entering the narrowing generates an audible click that indicates to a user that the action has been performed correctly. Thus, the audible click provides the user with a guarantee that the stopper has indeed broken simultaneously with the twisting of the cap through a given angle which corresponds to the travel of the lug between the first zone and the second zone.

Following this twisting, pulling the cap away from the body of the syringe causes the lug to slide in the disengagement opening or groove and then allows the cap to be released with the stopper coupled to it thereby providing access to the medicinal product contained in the syringe.

As a preference, the exterior part of the tip comprises two diametrically opposite snap-fastening lugs and the skirt comprising two diametrically opposite snap-fastening grooves or open-ended openings each collaborating with one of the two snap-fastening lugs.

According to one advantageous manufacturing variant, the tubular body, the connection tip with the stopper and the snap-fastening lug(s) are molded as one as a single one-piece component.

Advantageously, the one-piece component is produced in a synthetic material and obtained by molding. The material may be a plastic, advantageously polypropylene (PP).

The cap comprises at least one open-ended orifice intended for the passage of a sterilizing fluid. The orifice(s) in particular allow steam to easily access the parts that are to be sterilized, which are partly or fully covered by the cap. Thus, when sterilizing in wet heat, the steam from an autoclave passes through the orifice(s) in the cap and passes notably toward those surfaces of the connection tip that lie inside the volume delimited by the cap.

The cap may comprise a closure membrane stoppering the orifice in a fluid tight manner, as described and claimed in patent FR3084263B1. This closure membrane is advantageously made of Tyvek® and makes it possible to dispense with the use of a traditional exterior container (blister) which is equipped with a Tyvek®. This membrane allows water vapor (steam) to pass but ensures sterility in storage by preventing bacteria from passing. In other words, the membrane avoids any recontamination inside the cap once sterilization has been performed. This membrane may make it possible to dispense with the need for the currently existing blister packaging.

In place of the closure membrane, the pre-filled syringe may be packaged in an exterior container (blister) equipped with a Tyvek® which makes it possible to guarantee that the inside of the blister pack remains sterile.

According to an embodiment variant, the cap comprises an upper end wall from which the coupling portion projects in the form of a tube and is coupled to the stopper through complementing shapes. The sterilization orifice(s) is or are preferably made so that they open through this upper wall.

The stopper and/or the tubular coupling portion has or have complementing polygonal cross sections, and preferably a frustoconical overall shape. These shapes allow for optimal coupling and therefore provide a stronger guarantee that the stopper once ruptured is actually taken away by that part of the cap that is removed with the rest of the syringe.

The connection tip may be in the form of a cone of the Luer or Luer lock or NR Fit or EN Fit type or any other connection system incorporating a device for preventing errors with the other existing connection types thereby making it possible to avoid medication errors, to be connected notably to a needle or to an infusion or perfusion line or a catheter or an LAD or another commercially available connector, such as, for example, enteral/parenteral nutrition lines.

Thus, the invention essentially consists in an added-on cap fixed by snap-fastening/clip-fastening onto the connection tip or the tubular body of a pre-filled syringe and inside which there is fixed or coupled a breakable stopper the rupturing of which is guaranteed to be brought about by twisting at least part of the cap about the longitudinal axis of the syringe.

Having the entire cap or part of the cap held by the snap-fastening lug(s) during twisting makes it possible to ensure reliable breakage of the breakable zone of the stopper if the twisting action is performed correctly.

A breakage, that a user can observe visually, of breakable bridging pieces connecting part of the snap-fastened cap and another part to be removed with the stopper, or an audible click following a twist correctly performed by the user, indicates the aforementioned breakage to that user.

Of the numerous advantages of the invention, mention may be made of the following:
simple and effective opening of a pre-filled syringe by twisting with clean breakage and reliable removal of the stopper, which produces no unwanted particles and does not form any irregularities at the breakable zone;
prior to opening, effective fixing of the cap to the rest of the syringe by snap-fastening, ensuring reliable fluid tight packaging of the medicinal product contained in the syringe and preventing undesirable action on the cap that might lead to unwanted opening;
the guarantee of tamper resistance through the breakage of the breakable zone and the breakage of the breakable bridging pieces, when used.

Other advantages and features of the invention will become better apparent from reading the detailed description of exemplary embodiments of the invention which description is given by way of nonlimiting illustration with reference to the following figures.

### BRIEF DESCRIPTION OF THE DRAWINGS

FIG. 1 is a view in longitudinal section of a pre-filled syringe according to the prior art in the filled configuration prior to use.

FIG. 2 is a view in longitudinal section of part of a pre-filled syringe according to a first embodiment in the stoppered configuration with the cap fixed by snap-fastening prior to use.

FIG. 3 is a perspective view of part of a pre-filled syringe according to FIG. 2 before the fixing of the cap which is fixed by snap-fastening.

FIG. 4A is a perspective view of part of a pre-filled syringe according to FIGS. 2 and 3 illustrating the step of opening the syringe by twisting part of the cap.

FIG. 4B is a perspective view of part of a pre-filled syringe according to FIGS. 2 and 3 illustrating the opening of the syringe once this has been achieved.

FIG. 5 is a view in longitudinal section of part of a pre-filled syringe according to a second embodiment in the configuration in which it is stoppered with the cap fixed by snap-fastening prior to use.

FIG. 6 is a perspective view of the part of a syringe according to FIG. 5.

FIG. 7 is a perspective view of part of a pre-filled syringe according to FIGS. 5 and 6 prior to the fixing of the cap that is fixed by snap-fastening.

FIG. 8A is a perspective view of part of a pre-filled syringe according to FIGS. 5 to 7 illustrating the step of opening the syringe by twisting the cap.

FIG. 8B is a perspective view of part of a pre-filled syringe according to FIGS. 5 to 7 illustrating the step of opening the syringe by twisting the cap.

FIG. 8C is a perspective view of part of a pre-filled syringe according to FIGS. 5 to 7 illustrating the opening of the syringe by twisting the cap, once this has been achieved.

### DETAILED DESCRIPTION

The terms “lower”, “upper”, “top”, “bottom”, are to be understood with reference to a pre-filled syringe in a vertical configuration with the cap above the tubular body of the syringe.

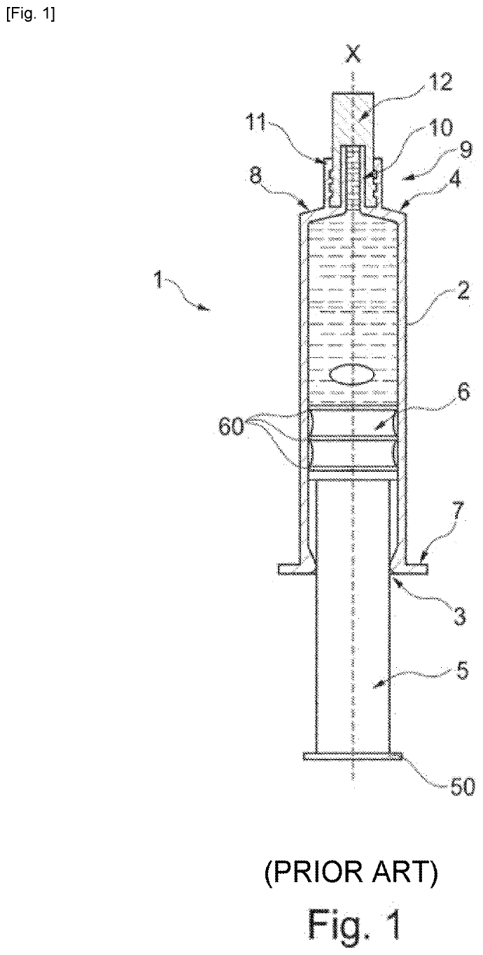

FIG. 1, which relates to a pre-filled syringe according to the prior art, has already been described in the preamble and will therefore not be commented upon hereinafter.

For the sake of clarity, an element in a syringe according to the prior art that is the same in a syringe according to the invention is designated by the same numerical reference.

Where they are not described, the elements of a pre-filled syringe according to the invention are analogous to those of a pre-filled syringe according to the prior art as shown in FIG. 1. In particular, a pre-filled syringe according to the invention comprises a plunger one of the ends of which forms a piston mounted to slide in the tubular body of the syringe, the plunger passing through the opening of one of the ends of the tubular body.

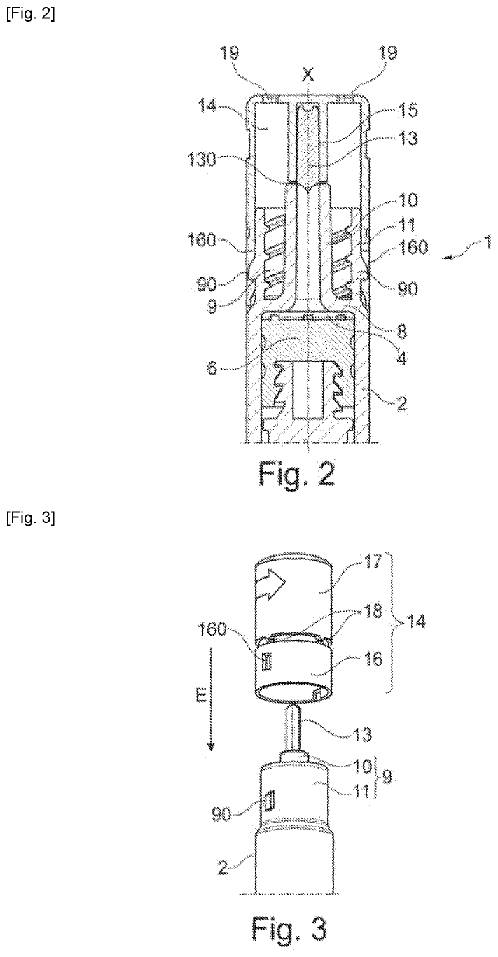

FIGS. 2 and 3 show a pre-filled syringe 1 according to a first embodiment of the invention. The syringe 1 of longitudinal axis X comprises a tubular body 2 one open end 4 of which incorporates a connection tip 9 of the Luer lock or Luer or NR Fit or EN Fit type (or any other specific form of connection developed for specific applications preventing errors with the other existing forms of connection).

The tip 9 comprises a tubular interior part 10 communicating with the opening in the transverse wall 8 of the tubular body 2 and an exterior part 11 which is internally threaded.

At least one snap-fastening lug 90, two diametrically opposite lugs in the example illustrated, projects toward the outside from the exterior part 11 of the tip 9.

A fluid tight stopper 13 is connected by a breakable zone 130 to the free end of the tubular part 10 of the tip 9. The breakable zone 130 is produced by an annular thinning of the material along the line along which the free end of the tubular part 10 meets the stopper 13. This means that these two parts can be separated, the thinned zone offering lower resistance to tearing or to rupture under the effect of twisting.

A cap 14 is mounted on the connection tip 9.

More specifically, the cap 14 comprises an interior portion 15 which is coupled to the fluid tight stopper 13 through complementing shapes, for example frustoconical overall shapes with hexagonal cross sections. In other words, the portion 15 of the cap 14 and the fluid tight stopper 13 have shapes that allow the stopper 13 to be force-fitted into said portion 15.

The cap 14 also comprises a portion forming a two-part skirt 16, 17, with the lower portion comprising at least one open-ended opening 160, two diametrically opposed openings in the example illustrated, which are each snap-fastened/clip-fastened onto one of the lugs 90 of the tip 9. This snap-fastening/clip-fastening thus secures the cap 14 to the tip 9 of the syringe. The two parts 16, 17 of the cap 14 are joined together, before the syringe is opened, by a plurality of breakable bridging pieces 18 which are equally annularly distributed about the longitudinal axis X.

The cap 14 further comprises at least one orifice 19, four equally annularly distributed orifices in the example illustrated, intended for the passage of a sterilizing fluid. When sterilizing using wet heat, the steam can thus infiltrate from outside the cap 14 to those zones of the tip 9 that are to be sterilized. The cap may comprise a closure membrane plugging all the orifices in a fluid tight manner, as described and claimed in patent FR3084263B1, making it possible to dispense with the use of a blister equipped with a Tyvek®.

In order to achieve simultaneous coupling between the tubular portion 15 and the stopper 13 and fixing of the cap 14 to the tip 9, an operator or an assembly machine brings the cap 14 onto the longitudinal axis X of the tip 9 and brings these two elements closer together in a translation movement as shown in FIG. 3 in the direction of the arrow E until the lugs 90 snap-fasten into the complementing openings 160 provided for that purpose.

The snap-fastening lugs 90 and the complementing open-ended openings 160 are configured to allow a twisting of at least part of the cap 14 about the longitudinal axis X to cause the breakable zone 130 to rupture and thereby allow the cap with the stopper to be physically separated from the tip.

Any other undesirable manual effort applied to the part of the cap, for example a pulling force, will not allow the cap to be opened. Specifically, the lugs 90 snap-fastened into the openings 90 mechanically prevent unwanted removal of the cap 14, which might be attempted notably by pulling on it.

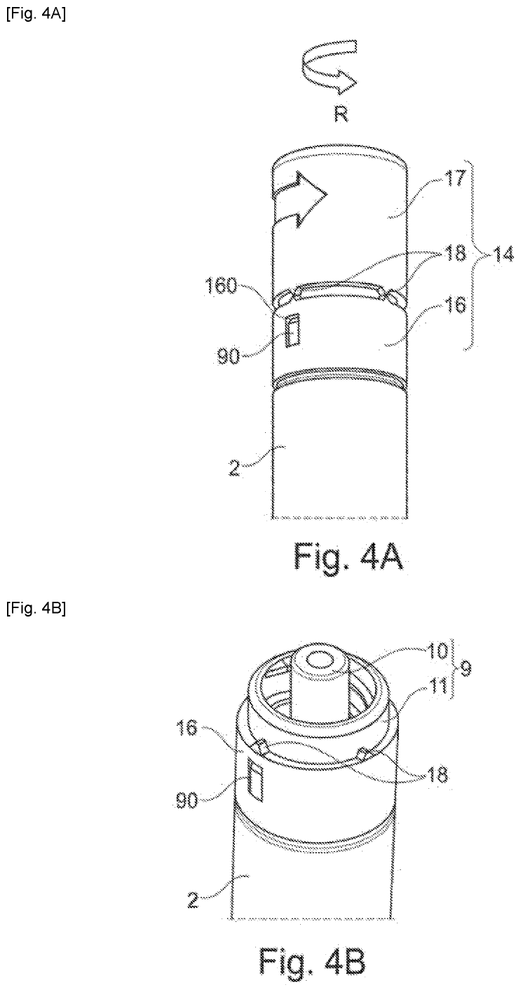

Thus, in order to open the syringe 1, a user manually twists the upper part of the cap 14 (FIG. 4A, arrow R) until the bridging pieces 18 rupture. This rupturing or breakage visually indicates to the user that the action has been performed correctly. If these bridging pieces 18 are already broken, the user knows that the syringe 1 has already been opened and that, therefore, the integrity of the syringe cannot be guaranteed.

The twisting simultaneously causes the stopper 13 to break.

Once this twisting has been performed, the user can proceed to pull on the cap 14, which opens the syringe because the upper part 17 of the cap 14 with the stopper 13 taken off and held inside it have been removed (FIG. 4B).

The lower part 16 of the cap 14 itself still remains snap-fastened to the tip 9.

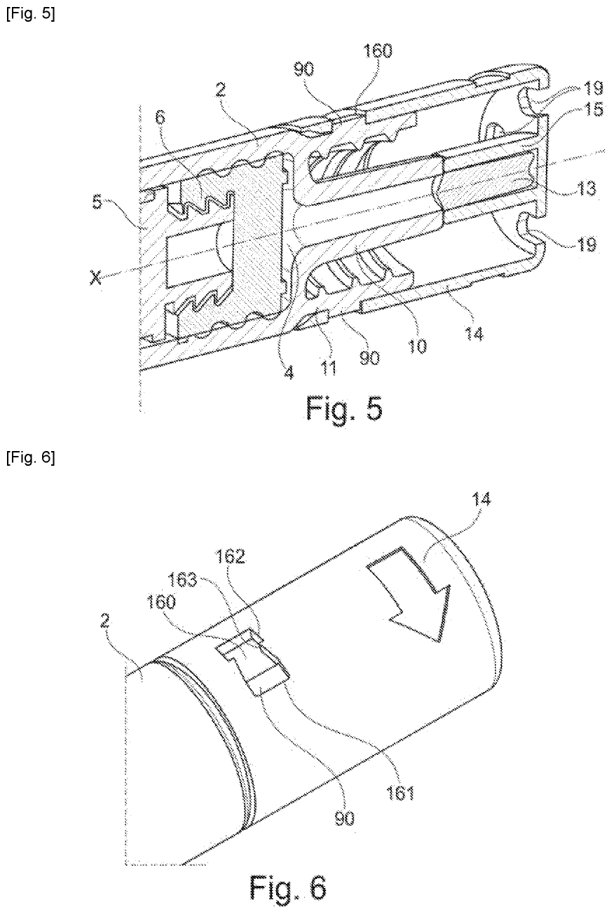

FIGS. 5 to 7 show a pre-filled syringe 1 according to a second embodiment of the invention. Here, the cap 14 consists of a single one-piece component.

Each snap-fastening open-ended opening 160 is delimited by two zones 161, 162 connected to one another by a narrowing 163. A disengagement groove 164 is formed in the continuation of the zone 162 as far as the lower edge of the skirt of the cap.

One of the two zones 161 is the one into which a lug 90 is snap-fastened.

The other of the two zones 162 is the one in which the lug 90 will become lodged when the cap 14 is twisted.

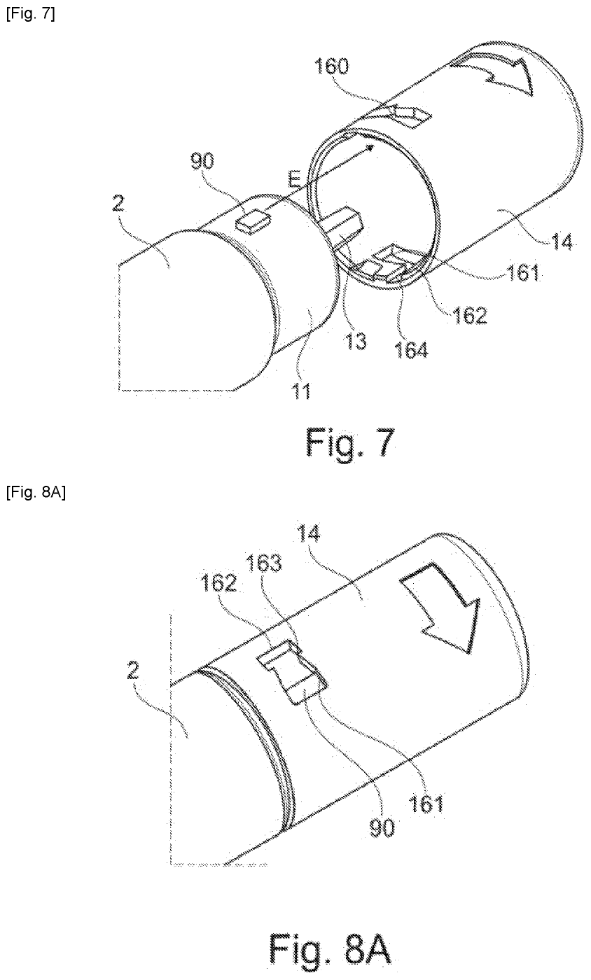

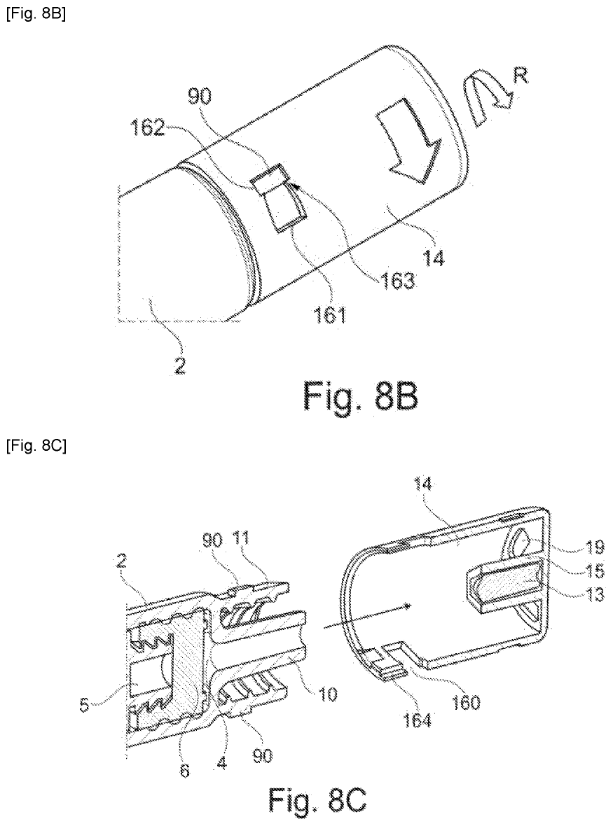

In order to achieve simultaneous coupling-together of the tubular portion 15 and the stopper 13 and the fixing of the cap 14 on the tip 9, an operator or an assembly machine brings the cap 14 onto the longitudinal axis X of the tip 9 and brings these two elements closer together in a translational movement as shown in FIG. 7 in the direction of the arrow E until the lugs 90 snap-fasten into those zones 161 of the complementing openings 160 that are provided for this purpose (FIG. 8A).

In order to open the syringe 1, a user manually twists the cap 14 (FIG. 8B, arrow R) until the lugs 90 come into abutment in the zone 162. The positioning of the lugs 90 visually indicates to the user that the breakage of the stopper 13 has indeed occurred.

Once this twisting action has been performed, the user can proceed to pull on the cap 14, which opens the syringe because the cap 14 with the stopper 13 taken off and held inside it are removed (FIG. 8C). Removal is possible because on pulling, each lug 90 slides from the zone 162 in a corresponding disengagement groove 164 until it becomes disengaged from the cap 14.

The narrowing 163 between the two zones 161, 162 is configured so that as the cap is twisted, the movement of the lug into the narrowing 163 generates an audible click indicating to a user that the action has been performed correctly.

In the embodiment in FIGS. 2 to 4B and 5 to 8C, the tubular body 2, the connection tip 9, the stopper 13 and the snap-fastening lugs 90 are made from a synthetic material in a single piece by molding.

Other variants and refinements may be envisioned without in any way thereby departing from the scope of the invention.

For example, although in all the embodiments illustrated, the lugs are produced on the periphery of the connection tip of the syringe and the open-ended openings into which the lugs are individually snap-fastened are produced on the periphery of the cap, it is conceivable to envision the reverse, which is to say with lugs on the periphery of the cap and corresponding snap-fastening grooves on the periphery of the connection tip.

1. A pre-filled syringe comprising:
a tubular body extending along a longitudinal axis, having two open ends and containing a fluid to be administered to a patient,
a plunger, one of the ends of which forms a piston, mounted for sliding in the tubular body, the plunger passing through the opening of one of the ends of the tubular body,
a connection tip comprising a tubular part communicating with the opening of another of the ends of the tubular body to create a passage for the fluid that is to be administered;
a fluid tight stopper comprising a breakable zone connected to the free end of the tubular part of the tip,
a cap comprising:
a fixing or coupling portion for fixing or coupling to the stopper,
a portion forming a skirt in dissociable form or molded as one with the fixing or coupling portion, and which extends as far as the tubular body; and
means for fixing at least part of the cap to the tubular body or the connecting tip, the means comprising at least one snap-fastening lug and at least one snap-fastening opening or groove collaborating in snap-fastening with the snap-fastening lug and being configured to allow a twisting of at least part of the cap about its longitudinal axis to cause the breakable zone to rupture thereby allowing the cap with the stopper to be physically separated from the tip.

2. The pre-filled syringe as claimed in claim 1, wherein
the connection tip comprises an exterior part, at an exterior periphery of the tubular part, comprising the at least one outwardly projecting snap-fastening lug; and
the skirt comprises the at least one open-ended snap fastening opening or groove collaborating in snap-fastening with the at least one lug.

3. The pre-filled syringe as claimed in claim 1, wherein
the skirt comprises an interior part comprising at least one inwardly projecting lug; and
the connection tip comprises an exterior part, at an exterior periphery of the tubular part, the at least one open-ended snap-fastening opening or groove collaborating in snap-fastening with the at least one lug.

4. The pre-filled syringe as claimed in claim 2, wherein the cap comprises:
two distinct parts one of them being in dissociable form or molded as one with the fixing or coupling portion and the other extending as far as the tubular body and incorporating the at least one opening or groove, and
at least one twist-breakable bridging piece, connecting the two parts some distance from one another so that the twisting of the part in dissociable form or molded as one with the fixing or coupling portion does not cause the other part, which is kept snap-fastened, to turn but causes simultaneous rupturing of the breakable zone and of the at least one bridging piece, the rupturing of the latter providing a user with a visual indication that an action has been performed correctly.

5. The pre-filled syringe as claimed in claim 2, wherein the cap is produced as a single one-piece component comprising:
the at least one snap-fastening groove or opening delimited by at least two zones, a first in which the snap-fastening of the lug occurs and at least a second in continuity of the first, and in which the lug becomes lodged when the cap is twisted; and
at least one disengagement open-ended opening or groove in the continuation of the second zone, the disengagement open-ended opening or groove being configured to allow physical separation of the cap with the stopper.

6. The pre-filled syringe as claimed in claim 5, wherein the at least one snap-fastening groove or opening comprises a single second zone allowing the cap to be twisted in just one direction of rotation.

7. The pre-filled syringe as claimed in claim 5, wherein the at least one snap-fastening groove or opening comprises a narrowing between the two zones, which narrowing is configured so that when the cap is twisted, an action of the lug entering the narrowing generates an audible click that indicates to a user that the action has been performed correctly.

8. The pre-filled syringe as claimed in claim 2, wherein the exterior part of the tip or the skirt comprises two diametrically opposite snap-fastening lugs and the skirt or the tip respectively comprises two diametrically opposite snap-fastening grooves or open-ended openings each collaborating with one of the two snap-fastening lugs.

9. The pre-filled syringe as claimed in claim 1, wherein the tubular body, the connection tip with the stopper and the snap-fastening lug(s) are molded as one as a single one-piece component.

10. The pre-filled syringe as claimed in claim 9, wherein the one-piece component is produced in a synthetic material and obtained by molding.

11. The pre-filled syringe as claimed in claim 1, wherein the cap comprises at least one open-ended orifice intended for the passage of a sterilizing fluid.

12. The pre-filled syringe as claimed in claim 1, wherein the cap comprises an upper end wall from which the coupling portion projects in the form of a tube and is coupled to the stopper through complementing shapes.

13. The pre-filled syringe as claimed in claim 12, the stopper and/or the tubular coupling portion having complementing polygonal cross sections.

14. The pre-filled syringe as claimed in claim 1, wherein the connection tip is in the form of a cone of a Luer or Luer lock or NR Fit or EN Fit type or any other connection system incorporating an error-prevention device to be connected to a needle or to an infusion or perfusion line or a catheter or an LAD or another connector.
